(19) United States
(12) Patent Application Publication
(10) Pub. No.: US 2021/0114957 A1
(43) Pub. Date: Apr. 22, 2021

(54) APPARATUS AND METHOD FOR PRODUCING METHANOL

(71) Applicant: Avocet IP Limited, Berwick-Upon-Tweed (GB)

(72) Inventors: James Robert JENNINGS, Yarm, North Yorkshire (GB); Glyn David SHORT, Hockessin, DE (US)

(21) Appl. No.: 16/464,347

(22) PCT Filed: Nov. 27, 2017
(86) PCT No.: PCT/EP2017/025345

§ 371 Date: May 28, 2019

(30) Foreign Application Priority Data
Nov. 26, 2016 (GB) 1620029.7

(51) Int. Cl. C07C 29/151; C12M 1/34; C12M 1/00; C01B 3/34

(52) U.S. Cl. CPC C07C 29/1518; C12M 41/30; C12M 21/12; C01B 2203/142; C01B 2203/0233; C01B 2203/0238; C01B 2203/061; C01B 3/34

(57) ABSTRACT

An apparatus is provided for producing methanol from organic material, characterized in that the apparatus includes

an anaerobic digestion arrangement for receiving the organic material and for anaerobically-digesting the organic material in oxygen-depleted conditions to generate methane gas; and
a chemical reaction arrangement for reacting the methane gas with water vapour and carbon dioxide in a stoichiometric condition (Eq. 4) between methane steam reforming and methane dry reforming to generate methanol.

The apparatus is operable to support a stoichiometric reaction as follows:

CO₂+3CH₄+2H₂O=4CH₃OH Eq. 4

The chemical reaction arrangement is operable to provide the stoichiometric condition (Eq. 4)

at a first stage for steam reforming at a pressure in a range of 10 Bar to 30 Bar, and at a temperature in a range of 750° C. to 950° C.; and
at a second stage of methanol synthesis at a pressure in a range of 50 Bar to 100 Bar, and at a temperature in a range of 200° C. to 250° C.

Optionally, a catalyst arrangement is employed for at least the second stage.

### TECHNICAL FIELD

The present disclosure relates to methods of producing methanol, for example to methods of producing methanol from organic waste material, for example agricultural organic waste. Moreover, the present disclosure also relates to apparatus that are operable to implement aforementioned methods. Furthermore, the present disclosure relates to computer program products comprising a non-transitory computer-readable storage medium having computer-readable instructions stored thereon, the computer-readable instructions being executable by a computerized device comprising processing hardware for executing aforementioned methods.

### BACKGROUND

In overview, methanol (CH₃OH) is a liquid fuel at room temperature (i.e. at circa 20° C.) that is storable in steel tanks, being relatively non-corrosive in nature. Methanol is not highly toxic, although a mere 30 cm³ to 100 cm³ quantity of methanol can be lethal if ingested. It is less dangerous than gasoline if inhaled, and far less toxic than two popular household cleaning fluids, namely trichloroethylene and carbon tetrachloride.

It is known that methanol is corrosive to certain materials in a vehicle's fuel system, for example aluminium components. Contemporary metal floats and synthetic cements employed in vehicle manufacture resist a solvent action exhibited by methanol. Iron and steel are quite immune to corrosion from methanol, as are also brass and bronze alloys.

Methanol is potentially a highly valuable energy carrier, because it can be combusted in contemporary combustion engines to provide mechanical power, and can also be oxidized in fuel cells to provide electrical power. Moreover, the oxidation of methanol results in the generation of carbon dioxide and water vapour that are regarded as benign to the environment.

Although methanol is a major, product of the petrochemicals industries with an annual tonnage well in excess of 100 million tonnes per annum, it has not found general significant use in transport, heating buildings and aviation because its volume-to-energy density is less than that of petrol, diesel oil and kerosene. Thus, for many industrial processes, methanol has not been used as extensively as possible.

With growing environmental concerns, despite considerable “unseen” pollution from nuclear power plants and similar industrial sites occurring, there is contemporary concern to recycle waste products from industry and farming to reduce their environmental impact, as the World struggles to try to achieve a greater degree of long-term sustainability in its commercial activities. Agricultural waste is potentially an environmental issue that has caused concern more recently. In particular, it is desirable to convert agricultural waste that is otherwise a cost overhead into a valuable commercial by-product.

### SUMMARY

The present disclosure seeks to provide an improved method of generating methanol, for example from biological waste, for example agricultural waste.

Moreover, the present disclosure seeks to provide an improved apparatus for implementing aforementioned improved methods.

According to a first aspect, there is provided method of using an apparatus for producing methanol in a continuous manner from organic material, characterized in that the method includes:
(i) receiving the organic material in an anaerobic digestion arrangement, and anaerobically-digesting the organic material in oxygen-depleted conditions to generate a gas comprising methane and carbon dioxide;
(ii) simultaneously reacting the gas with water vapour and carbon dioxide in a stoichiometric condition of reaction

CO₂+3CH₄+2H₂O=4CH₃OH
wherein molar ratio of methane to carbon dioxide is to be in a range from 2.5:1 to 4.0:1, and molar ratio of methane to water is to be in a range from 2.5:2 to 4.0:2 between regimes of methane steam reforming and methane dry reforming within at least one vessel to generate a synthesis gas in a chemical reaction arrangement, characterized in that the chemical reaction arrangement is operated at a first stage for steam reforming at a pressure in a range of 10 Bar to 30 Bar, and at a temperature in a range of 750° C. to 950° C.; and
(iii) converting the synthesis gas to methanol in the chemical reaction arrangement, characterized in that the chemical reaction arrangement is operated at a second stage of methanol synthesis at a pressure in a range of 50 Bar to 150 Bar, and at a temperature in a range of 200° C. to 250° C.

The aspects of the disclosed embodiments are of advantage in that operating substantially at the stoichiometric condition (Eq. 4) allows for highly efficient production of methanol, based on biogas supplied from an anaerobic digester supplied for organic material, for example organic agricultural waste. Moreover, embodiments of the present invention advantageous in terms of significantly reducing amount of bi-products formed during production of methanol despite of operating the chemical reaction arrangement at low temperature and using low cost and/or less active catalysts.

Optionally, the method includes maintaining the stoichiometric condition using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, method includes using a renewable energy source for providing operating power to the chemical reaction arrangement.

Optionally, the method includes operating the chemical reaction arrangement to employ a catalyst arrangement including nickel-alumina, nickel foil, coper and/or platinum catalysts.

According to a second aspect, there is provided an apparatus for producing methanol in a continuous manner from organic material, characterized in that the apparatus includes:
(i) an anaerobic digestion arrangement for receiving the organic material and for anaerobically-digesting the organic material in oxygen-depleted conditions to generate methane gas; and
(ii) a chemical reaction arrangement for:
   a) simultaneously reacting the methane gas with water vapour and carbon dioxide in a stoichiometric condition of reaction

CO₂+3CH₄+2H₂O=4CH₃OH
wherein molar ratio of methane to carbon dioxide is to be in a range from 2.5:1 to 4.0:1, and molar ratio of methane to water is to be in a range from 2.5:2 to 4.0:2 between regimes of methane steam reforming and methane dry reforming within at least one vessel to generate a synthesis gas in the chemical reaction arrangement, characterized in that the chemical reaction arrangement is operated at a first stage for steam reforming at a pressure in a range of 10 Bar to 30 Bar, and at a temperature in a range of 750° C. to 950° C.; and
b) converting the synthesis gas to methanol in the chemical reaction arrangement, characterized in that the chemical reaction arrangement is operated at a second stage of methanol synthesis at a pressure in a range of 50 Bar to 150 Bar, and at a temperature in a range of 200° C. to 250° C.

Optionally, the stoichiometric condition is maintained using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, the apparatus includes a renewable energy source for providing operating power to the chemical reaction arrangement.

Optionally, the chemical reaction arrangement is operable to employ a catalyst arrangement including, nickel, nickel-alumina, nickel foil, copper and/or platinum catalysts.

According to a third aspect, there is provided a computer program product comprising a non-transitory computer-readable storage medium having computer-readable instructions stored thereon, the computer-readable instructions being executable by a computerized device comprising processing hardware for executing a method of the first aspect.

It will be appreciated that features of the invention are susceptible to being combined in various combinations without departing from the scope of the invention as defined by the appended claims.

### BRIEF DESCRIPTION OF THE DIAGRAMS

Embodiments of the present disclosure will now be described, by way of example only, with reference to the following diagrams wherein:

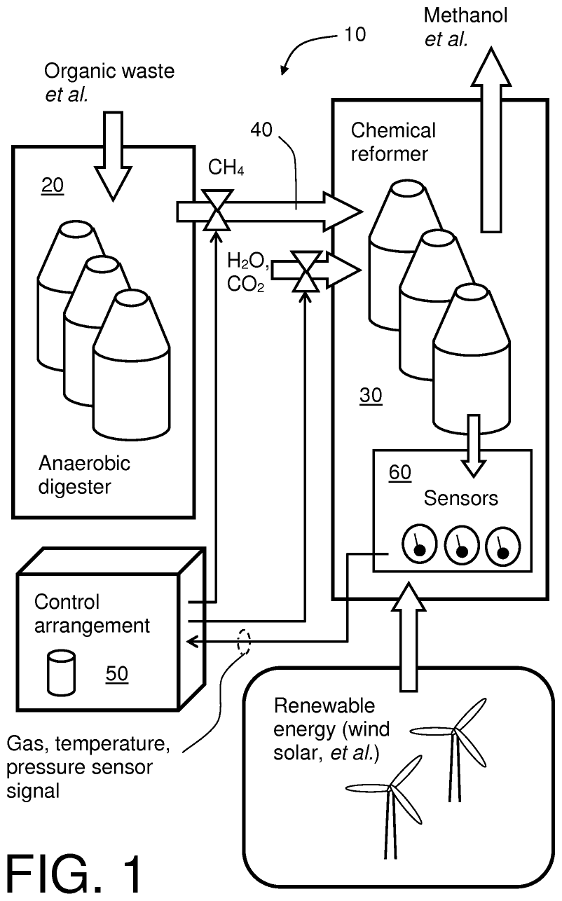

FIG. 1 is an illustration of an apparatus for producing methanol pursuant to the present disclosure; and

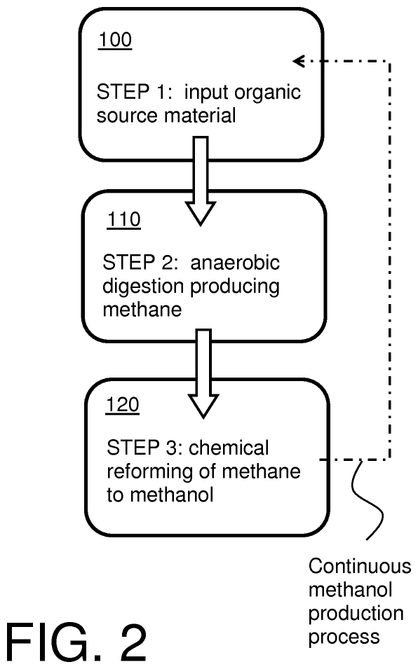

FIG. 2 is an illustration of steps of a method of producing methanol using the apparatus of FIG. 1.

In the accompanying diagrams, an underlined number is employed to represent an item over which the underlined number is positioned or an item to which the underlined number is adjacent. When a number is non-underlined and accompanied by an associated arrow, the non-underlined number is used to identify a general item at which the arrow is pointing.

### DETAILED DESCRIPTION OF EMBODIMENTS

According to a first aspect, there is provided an apparatus for producing methanol from organic material, characterized in that the apparatus includes:
(i) an anaerobic digestion arrangement for receiving the organic material and for anaerobically-digesting the organic material in oxygen-depleted conditions to generate methane gas; and
(ii) a chemical reaction arrangement for reacting the methane gas with water vapour and carbon dioxide in a stoichiometric condition (Eq. 4) between methane steam reforming and methane dry reforming to generate a synthesis gas more suited to the production of methanol, characterized in that the chemical reaction arrangement is operable to provide the stoichiometric condition (Eq. 4):
   a) at a first stage for steam reforming at a pressure in a range of 10 Bar to 30 Bar, and at a temperature in a range of 750° C. to 950° C.; and
   b) at a second stage of methanol synthesis at a pressure in a range of 50 Bar to 150 Bar, and at a temperature in a range of 200° C. to 250° C.

Optionally, in the apparatus, the stoichiometric condition is maintained using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, the apparatus includes a renewable energy source for providing operating power to the chemical reaction arrangement.

Optionally, in the apparatus, the chemical operating arrangement is operable to employ a catalyst arrangement including nickel-alumina, nickel foil, copper-zinc-alumina and/or platinum catalysts.

Optionally, the apparatus is operable to produce methanol in a continuous manner.

According to a second aspect, there is provided a method of using an apparatus for producing methanol from organic material, characterized in that the method includes:
(i) receiving the organic material in an anaerobic digestion arrangement, and anaerobically-digesting the organic material in oxygen-depleted conditions to generate a gas comprising methane and carbon dioxide; and
(ii) reacting the methane gas with water vapour and carbon dioxide in a stoichiometric condition (Eq. 4) between methane steam reforming and methane dry reforming to generate a synthesis gas in a chemical reaction arrangement, characterized in that the chemical reaction arrangement is operated at a first stage for steam reforming at a pressure in a range of 10 Bar to 30 Bar, and at a temperature in a range of 750° C. to 950° C.; and
(iii) converting the synthesis gas to methanol in the chemical reaction arrangement, characterized in that the chemical reaction arrangement is operated at a second stage of methanol synthesis at a pressure in a range of 50 Bar to 150 Bar, and at a temperature in a range of 200° C. to 250° C.

Optionally, the method includes maintaining the stoichiometric condition using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

Optionally, the method includes using a renewable energy source for providing operating power to the chemical reaction arrangement.

Optionally, the method includes operating the chemical reaction arrangement to employ a catalyst arrangement including nickel-alumina, nickel foil, copper and/or platinum catalysts.

Optionally, the method includes operating the apparatus to produce methanol in a continuous manner.

According to a third aspect, there is provided a computer program product comprising a non-transitory computer-readable storage medium having computer-readable instructions stored thereon, the computer-readable instructions being executable by a computerized device comprising processing hardware for executing a method of the first aspect.

In overview, the present disclosure is concerned with a method of processing organic waste in an anaerobic digestion arrangement to provide methane gas, and then to reform the methane gas to generate corresponding methanol. Energy for implementing the method beneficially is provided from renewable energy resources, for example solar cells, heliostats, wind turbines, hydroelectric turbines (for example, micro-turbines inserted into small streams and rivers).

The method includes a concurrent combination of:
(i) steam reforming of methane to generate methanol and an excess of hydrogen; and
(ii) dry reforming of methane with carbon dioxide to generate methanol and an excess of carbon monoxide,

wherein a combination of (i) and (ii) in a correct stochiometric proportion is operable to produce a gas mixture that is optimal for purposes of methanol synthesis.

Chemical reactions associated with (i) and (ii) will next be described in greater detail.

In a first stage, organic waste (for example, livestock waste, animal slurry, cellulose plant-harvest waste, denatured fruit and vegetables and similar that are unsuitable for sale for human consumption or for animal feed) and/or organic crop material (for example, maize) is provided to an anaerobic digester arrangement wherein, in an oxygen-depleted environment, microorganisms are operable to convert the organic waste and/or organic crop material into methane and other reaction by-products.

In the anaerobic digester arrangement, there is employed a collection of processes by which microorganisms break down biodegradable material in the absence of oxygen. Such a process is contemporarily used for industrial or domestic purposes to manage waste, or to produce fuels. The processes are akin, in many respects, to fermentation that is used industrially to produce food and drink products. It will be appreciated that anaerobic digestion occurs naturally in some soils and in lake and oceanic basin sediments, where it is usually referred to as “anaerobic activity”. This is the source of marsh gas methane as discovered by a scientist Volta in year 1776.

In the aforementioned anaerobic digester arrangement, there occurs in operation a digestion process that begins with bacterial hydrolysis of input materials provided to the anaerobic digester arrangement, for example agricultural waste as aforementioned. Insoluble organic polymers, such as carbohydrates, are broken down to soluble derivatives (including sugars and amino acids) that become available for other bacteria that are present in the anaerobic digester arrangement. Thereafter, acidogenic bacteria then convert the sugars and amino acids into carbon dioxide gas, hydrogen gas, ammonia gas and organic acids. Moreover, these acidogenic bacteria convert these resulting organic acids into acetic acid, along with additional ammonia gas, hydrogen gas, and carbon dioxide gas. Finally, methanogens convert such gaseous products to methane and carbon dioxide. Thus, such methanogens, for example methanogenic archaea populations, play an indispensable role in anaerobic wastewater treatments that are feasible to achieve using the aforementioned anaerobic digester arrangement.

The anaerobic digestion arrangement is operable to function as a source of renewable energy, for example for producing biogas, consisting of a mixture of methane, carbon dioxide and traces of other trace gases. This biogas can be used directly as fuel, in combined heat and power gas engines or upgraded to natural gas-quality bio-methane. There is also generated from the anaerobic digestion arrangement a nutrient-rich digestate that can be used as a fertilizer.

In practice, the anaerobic digestion arrangement includes at least one closed vessel, for example fabricated from welded steel sheet, and is provided with a screw-feed arrangement for introducing, for example in a continuous manner, the aforementioned organic waste and/or organic crop material into the at least one closed vessel. Anaerobic digestion processes occurring within the at least one vessel result in an excess gaseous pressure to arise within the at least one vessel, wherein biogas can be selectively vented from the at least one vessel to provide biogas feedstock to a subsequent process. Beneficially, a screw-feed arrangement is used to remove digestate, for example in a continuous manner, from a lower region of the at least one vessel.

In embodiments of the present disclosure, the biogas feedstock is provided to a chemical reforming arrangement that will next be described in greater detail. The chemical reforming arrangement is beneficially implemented as a two-stage process involving:
(i) a first stage of steam reforming; and
(ii) a second stage of methanol synthesis.

The stages are optionally implemented in a single reaction vessel. Alternatively, the stages are optionally implemented in two or more reaction vessels. Beneficially, when two or more reaction vessels are employed, a first reaction vessel is operable to accommodate in operation steam reforming and a second reaction vessel is operable to accommodate in operation methanol synthesis.

A plurality of controllable gas feeds is provided to the at least one reaction vessel, for example two or more reaction vessels, including a gas feed for the aforementioned biogas from the anaerobic digestion arrangement. The at least one reaction vessel is provided with a gas sensing arrangement, for example implemented using one or more infrared radiation absorption gas analyzers and/or electrochemical gas analyzers, for measuring a stoichiometry of gases present in operation within the at least in one reaction vessel. Optionally, the at least one reaction vessel is provided with a catalyst arrangement, for example for the second stage, for example for both first and second stages, for example a metal mesh arrangement (for example fabricated from Nickel Alumina, Nickel foil, Platinum, Copper or similar), and a source of heat.

The source of heat is optionally supplied from renewable energy resources, for example spatially geographical local to the chemical reforming arrangement (for example, as would be appropriate for off-grid implementations of embodiments of the present disclosure when implemented in a rural environment, for example when operated in rural Latin America, rural India, rural Middle East, on isolated islands and such like).

For the first stage of steam reforming, there is utilized an internal pressure in the at least one vessel in a range of 5 Bar to 50 Bar, and more optionally in a range of 10 Bar to 30 Bar. Moreover, for the first stage of steam forming, the at least one reaction vessel is, for example, optionally operated having an internal operating temperature in a range of 300° C. to 1200° C., more optionally an internal operating temperature in a range of 750° C. to 950° C. When implementing the first stage of steam forming, there is beneficially provided an excess of hydrogen (H₂) for the steam reforming reaction.

For the second stage of methanol synthesis, there is utilized an internal pressure in the at least one vessel in a range of 30 Bar to 150 Bar, and more optionally in a range of 50 Bar to 100 Bar. Moreover, for the second stage of methanol synthesis, the at least one reaction vessel is, for example, optionally operated having an internal operating temperature in a range of 150° C. to 300° C., more optionally an internal operating temperature in a range of 200° C. to 250° C. Preferably, operating temperatures in excess of 260° C. are avoided, as they tend to result in a formation of metallic nanoparticles, for example copper nanoparticles, on catalyst surfaces that can be detrimental to throughput of synthesis of methanol during the second stage. The second stage, in operation results in an excess of carbon dioxide (CO₂) that is reacted with excess hydrogen (H₂) from the first stage.

A processor-based control arrangement is provided and is operable to monitor and control the stoichiometric composition of gases within the at least one reaction vessel (for example a single vessel, two vessels, and so forth, as aforementioned) the internal operating temperature of the at least one reaction vessel, the internal pressure of the at least one reaction vessel, gas mixing occurring within the at least one reaction vessel (for example flows of steam, biogas and carbon dioxide (for example a degree of turbulence in mixing), and optionally a temperature of a catalyst arrangement present within the at least one reaction vessel.

Chemical reactions occurring within the at least one reaction vessel are primarily concerned with converting biogas provided from the anaerobic digestion arrangement, namely principally methane, into methanol. Beneficially, the at least one reaction vessel is heated with energy supplied from renewable energy sources, for example wind turbine, solar panels and so forth.

In methane steam reforming processes, as employed for the first stage, there is generated an excess of hydrogen (H₂), relative to the amount of carbon oxides generated for methanol synthesis; such a methane steam reforming process is represented by Equation 1 (Eq. 1):

CH₄+H₂O=CO+3H₂=CH₃OH+H₂ Eq. 1

However, in methane dry reforming processes, as employed for the second stage, there is produced a gas that is deficient in hydrogen (H₂) for methanol synthesis, relative to the amount of residual carbon oxides; such a methane dry reforming process is represented by Equation 2 (Eq. 2):

2CH₄+2CO₂=4CO+4H₂=2CH₃OH+2CO Eq. 2

The aforementioned at least one reaction vessel of the chemical reforming arrangement employs a combination of operating conditions that lie between regimes represented by Equation 1 (Eq. 2) and Equation 2 (Eq. 2). A combination of the two regimes represented by Equation 1 (Eq. 2) and Equation 2 (Eq. 2) in a correct proportion is operable to produce a gas mixture that is just optimal for purposes of methanol synthesis.

Thus, in the chemical reforming arrangement, the following two reactions (Eqs. 5 & 6) pertain simultaneously within the at least one vessel (Eqs. 3A, 3B), for example two or more vessels:

CO₂+3H₂=CH₃OH+H₂O Eq. 3A

3CH₄+3H₂O=3CH₃OH+3H₂ Eq. 3B

Thus, when the stoichiometry of gaseous reactants present in operation within the at least one reaction vessel is appropriately controlled, there is derived by addition that a chemical reaction as provided by Equation 4 (Eq. 4) is achieved:

CO₂+3CH₄+2H₂O=4CH₃OH Eq. 4

When stoichiometry is achieved, an amount of hydrogen (H2) and carbon dioxide generated (CO2) at the first and second stages is substantially matched, for example to within at least 10%, more optionally to within at least 5%, and yet more optionally to within at least 1%.

From the foregoing, it will be appreciated that if biogas generated by the anaerobic digestion arrangement is only slightly upgraded from its raw state of circa 60% methane and 40% carbon dioxide to exactly 75% methane and 25% methane, then steam reforming with an appropriate excess of steam is capable of producing an exactly stoichiometric synthesis gas required for efficient methanol manufacture. Appropriate reaction conditions are required, as described in the foregoing.

In an exemplary embodiment, the apparatus for producing methanol from organic material may include an anaerobic digestion arrangement for receiving the organic material and for anaerobically-digesting the organic material in oxygen-depleted conditions to generate a methane-containing AD gas; a chemical reaction arrangement for reacting the methane gas with water vapour and carbon dioxide in a stoichiometric condition (Eq. 4) between methane steam reforming and methane dry reforming to generate methanol synthesis gas; and a methanol synthesis arrangement for converting the methanol synthesis gas to methanol. Additionally in this embodiment, the chemical reaction arrangement of the apparatus may be operable to provide the stoichiometric condition (Eq. 4). Further, at the first stage for steam reforming the stoichiometric conditions may include but not limited to a pressure in a range of 10 Bar to 30 Bar, and a temperature in a range of 750° C. to 950° C. Furthermore, at the second stage of methanol synthesis the stoichiometric conditions may include but not limited to a pressure in a range of 50 Bar to 150 Bar, and a temperature in a range of 200° C. to 250° C. In practice, use of high temperature in the first stage for steam reforming the stoichiometric conditions is advantageous in terms of higher rate of reaction and removal of impurities present in feed received from the anaerobic digestion arrangement

In another embodiment, the apparatus for producing methanol from organic material may further include a methanol reformer for converting traces of Methane into Methanol received from purge stream of the chemical reaction arrangement. In this embodiment, the methanol reformer may include less exotic alloys/less active alloys as catalysts for converting traces of Methane into Methanol received from purge stream of chemical reaction arrangement. In practice, use of less exotic alloys/less active alloys as catalysts is advantageous in terms of reducing loss of methane due to recycling of the purge gasses.

In yet another embodiment, the chemical reaction arrangement of the apparatus may be operable to provide the stoichiometric condition (Eq. 4). In example, at the first stage for steam reforming the stoichiometric conditions may include but not limited to a pressure in a range of 10 Bar to 30 Bar, and a temperature in a range of 750° C. to 950° C. Further, at the second stage of methanol synthesis the stoichiometric conditions may include but not limited to a pressure in a range of 50 Bar to 150 Bar, and a temperature in a range of 200° C. to 250° C. In practice, use of less exotic alloys/less active alloys as catalyst at the second stage is advantageous in terms of reducing loss of methane due to recycling of the purge gasses and high yield of methanol.

In still another embodiment, the catalysts may include but not limited to nickel-alumina, nickel foil, copper and/or platinum.

In other exemplary embodiment, the method of using an apparatus for producing methanol from organic material may include receiving the organic material at an anaerobic digestion arrangement and anaerobically-digesting the organic material in oxygen-depleted conditions to generate methane gas, and reacting the methane gas with water vapour and carbon dioxide in a stoichiometric condition (Eq. 4) between methane steam reforming and methane dry reforming to generate methanol in the chemical reaction arrangement.

### DETAILED DESCRIPTION OF DRAWINGS

Referring to FIG. 1, there is shown an illustration of an apparatus for producing methanol purusant to the present disclosure. The apparatus is indicated generally by 10, and includes an anaerobic digestion arrangement 20 and a chemical reforming arrangement 30, wherein a biogas feed pipe arrangement 40 is operable to provide a flow of methane gas, in operation from the anaerobic digestion arrangement 20 to the chemical reforming arrangement 30. The anaerobic digestion arrangement 20 includes one or more anaerobic digestion vessels that are operable to provide for microorganism-based digestion of organic waste and/or organic materials under oxygen-depleted reaction conditions; the one or more anaerobic digestion vessels are, for example fabricated from seam-welded formed steel sheet, or similar. Moreover, the chemical reforming arrangement 30 includes one or more chemical reaction vessels, for example fabricated from seam-welded formed steel sheet, or similar; the one or more chemical reaction vessels are operable to accommodate the aforementioned first and second stages. Moreover, the apparatus 10 further includes a control arrangement 50 for controlling admission of gas components to an internal region of at least one reaction vessel of the chemical reforming arrangement 30, for example admission in operation of steam carbon dioxide and biogas into the at least one reaction vessel. Furthermore, a gas sensing arrangement 60, as described in the foregoing, is coupled to the at east one reaction vessel of the chemical reforming arrangement 30; the gas sensing arrangement 60 provides sensed gas concentration measurements (for example, p.p.m. concentration of carbon dioxide (CO₂) present in the at least one reaction vessel, p.p.m. concentration of methane (CH₄) present in the at least one reaction vessel, p.p.m. concentration of methanol (CH₃OH) present in the at least one reaction vessel, p.p.m. concentration of carbon monoxide (CO) present in the at least one reaction vessel, p.p.m. concentration of hydrogen (H₂) present in the at least one reaction vessel, p.p.m. concentration of water vapour (H₂O) present in the at least one reaction vessel) to the control arrangement 50 that employs an algorithm to control the admission of gas components to an internal region of at least one reaction vessel of the chemical reforming arrangement 30, for example to achieve a substantially stoichiometric reaction as aforementioned.

Referring next to FIG. 2, there is shown a method of operating the apparatus 10 of FIG. 1. In a first step S1 100 of the method, the method includes supplying organic material, for example agricultural waste, to the anaerobic digestion arrangement 20. In a second step S2 110 of the method, the method includes anaerobically digesting the supplied organic material to generate biogas, primarily methane. In a third step S3 120, the method includes using the control arrangement 50 to receive signals from the gas sensing arrangement 60 indicative of gas component concentrations present in the one or more chemical reaction vessels of the chemical reforming arrangement 30, to apply values corresponding to the received signals to a stochiometry control algorithm executed upon processing hardware of the control arrangement 50, to generate control signals from the stochiometry control algorithm and to apply the control signals to the biogas feed pipe arrangement 40 and to other sources of gases (for example, a carbon dioxide generator, a steam generator) to maintain an operating stochiometry within the one or more chemical reaction vessels (to maintain in operation a reaction condition as described by Equation 4 (Eq. 4). In a fourth step S4 130, the method includes extracting (for example, via a process of selective condensation) methanol from the one or pre chemical reaction vessels. The steps S1 to S4 are beneficially performed concurrently so that the apparatus 10 is capable of continuously generating methanol from organic waste and similar organic materials.

Modifications to embodiments of the invention described in the foregoing are possible without departing from the scope of the invention as defined by the accompanying claims. Expressions such as “including”, “comprising”, “incorporating”, “consisting of”, “have”, “is” used to describe and claim the present invention are intended to be construed in a non-exclusive manner, namely allowing for items, components or elements not explicitly described also to be present. Reference to the singular is also to be construed to relate to the plural. Numerals included within parentheses in the accompanying claims are intended to assist understanding of the claims and should not be construed in any way to limit subject matter claimed by these claims.

1-11. (canceled)

12. A method of using an apparatus for producing methanol in a continuous manner from organic material, wherein the method comprises:
(i) receiving the organic material in an anaerobic digestion arrangement, and anaerobically-digesting the organic material in oxygen-depleted conditions to generate a gas comprising methane and carbon dioxide;
(ii) simultaneously reacting the gas with water vapour and carbon dioxide in a stoichiometric condition of reaction
CO₂+3CH₄+2H₂O=4CH₃OH
wherein molar ratio of methane to carbon dioxide is to be in a range from 2.5:1 to 4.0:1, and molar ratio of methane to water is to be in a range from 2.5:2 to 4.0:2 between regimes of methane steam reforming and methane dry reforming within at least one vessel to generate a synthesis gas in a chemical reaction arrangement, characterized in that the chemical reaction arrangement is operated at a first stage for steam reforming at a pressure in a range of 10 Bar to 30 Bar, and at a temperature in a range of 750° C. to 950° C.; and
(iii) converting the synthesis gas to methanol in the chemical reaction arrangement, characterized in that the chemical reaction arrangement is operated at a second stage of methanol synthesis at a pressure in a range of 50 Bar to 150 Bar, and at a temperature in a range of 200° C. to 250° C.

13. The method of claim 12, wherein the method comprises maintaining the stoichiometric condition using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

14. The method of claim 12, wherein the method comprises using a renewable energy source for providing operating power to the chemical reaction arrangement.

15. The method of claim 12, wherein the method comprises operating the chemical reaction arrangement to employ a catalyst arrangement including nickel-alumina, nickel foil, coper and/or platinum catalysts.

16. An apparatus for producing methanol in a continuous manner from organic material, wherein the apparatus comprises:
(i) an anaerobic digestion arrangement for receiving the organic material and for anaerobically-digesting the organic material in oxygen-depleted conditions to generate methane gas; and
(ii) a chemical reaction arrangement for:
a) simultaneously reacting the methane gas with water vapour and carbon dioxide in a stoichiometric condition of reaction
CO₂+3CH₄+2H₂O=4CH₃OH
wherein molar ratio of methane to carbon dioxide is to be in a range from 2.5:1 to 4.0:1, and molar ratio of methane to water is to be in a range from 2.5:2 to 4.0:2 between regimes of methane steam reforming and methane dry reforming within at least one vessel to generate a synthesis gas in the chemical reaction arrangement (30), characterized in that the chemical reaction arrangement (30) is operated at a first stage for steam reforming at a pressure in a range of 10 Bar to 30 Bar, and at a temperature in a range of 750° C. to 950° C.; and
b) converting the synthesis gas to methanol in the chemical reaction arrangement (30), characterized in that the chemical reaction arrangement (30) is operated at a second stage of methanol synthesis at a pressure in a range of 50 Bar to 150 Bar, and at a temperature in a range of 200° C. to 250° C.

17. The apparatus of claim 16, wherein the stoichiometric condition is maintained using a control arrangement, provided in operation with temperature sensing signals and gas component sensing signals indicative of operating conditions within the chemical reaction arrangement, for controlling rates of supply of the methane gas, water vapour and carbon dioxide into the chemical reaction arrangement.

18. The apparatus of claim 16, wherein the apparatus comprises a renewable energy source for providing operating power to the chemical reaction arrangement.

19. The apparatus of claim 16, wherein the chemical reaction arrangement is configured to employ a catalyst arrangement including, nickel, nickel-alumina, nickel foil, copper and/or platinum catalysts.
